(19) United States
(12) Patent Application Publication
(10) Pub. No.: US 2018/0038863 A1
(43) Pub. Date: Feb. 8, 2018

(54) COMMON CAPTURE CELL SEPARATIONS DONE VIA SIMULTANEOUS INCUBATION OF COMPONENTS

(71) Applicant: BIOMAGNETIC SOLUTIONS LLC, State College, PA (US)

(72) Inventors: Paul A. Liberti, Naples, FL (US); Todor R. Khristov, State College PA, PA (US)

(21) Appl. No.: 15/553,707

(22) PCT Filed: Feb. 25, 2016
(86) PCT No.: PCT/US16/19535

§ 371 Date: Aug. 25, 2017

Related U.S. Application Data
(60) Provisional application No. 62/121,259, filed on Feb. 26, 2015.
(60) Provisional application No. 62/148,133, filed on Apr. 15, 2015.
(60) Provisional application No. 62/157,601, filed on May 6, 2015.
(60) Provisional application No. 62/174,687, filed on Jun. 12, 2015.

(51) Int. Cl. G01N 33/569; G01N 33/543; C12N 5/0783

(52) U.S. Cl. CPC G01N 33/56972; C12N 5/0636; G01N 33/54326; G01N 33/54346; C12N 2501/998; G01N 2333/7051; G01N 2333/70521

(57) ABSTRACT

Methods are provided for the rapid and efficient separation of target bioentities.

### CROSS-REFERENCE TO RELATED APPLICATIONS

This application claims priority under 35 U.S.C. section 119 (e) to U.S. Provisional Application Nos. 62/121,259 filed Feb. 26, 2015; 62/157,601 filed May 6, 2015; and 62/174,687 filed Jun. 12, 2015, the entire disclosures of each of the aforementioned applications being incorporated herein by reference as though set forth in full.

### FIELD OF THE INVENTION

This invention relates to improvements in the labeling and/or separations of targeted entities where particles large or small, e.g., magnetic nanoparticles, are used to bind to some entity of interest, thus allowing for the entity to be manipulated or retrieved with an appropriate method. It also relates to improvements and simplifications in the performance of separations that are done by indirect labeling processes where first cells or other target entities are labeled with a specific labeling agent, e.g., an antibody, and then labeled with a common capture particle that binds to antibody that is cell or entity bound. The invention discloses novel methods and means for improving such processes that make them easier to perform, minimizes required time, and significantly reduces the cost of such processes due to a significant reduction in the amount of reagents normally required for such processes. Further, the methods described herein are adaptable to automated separations requiring fewer processing steps.

### BACKGROUND OF THE INVENTION

A number of publications and patent documents are cited throughout the specification in order to describe the state of the art to which this invention pertains. Each of these citations is incorporated herein by reference as though set forth in full.

There are numerous manufacturing, analytical and laboratory processes and procedures, which involve specific binding pair interactions. Many laboratory and clinical procedures are based on such interactions, referred to as ‘bio-specific’ affinity reactions. Such reactions are commonly used in diagnostic testing of biological samples, or for the separation of a spectrum of target substances, especially biological entities such as cells, viruses, proteins, nucleic acids and the like. It is important in practice to perform the specific binding pair interactions as quickly and efficiently as possible. The effectiveness of such reactions depends on classical chemical variables such as temperature, concentration and binding affinity of pair members for one another. The use of high binding affinity pairs is important, particularly when the concentration of one of the specific binding pair members to be isolated is in extremely low concentrations, as often is the case in biological systems.

Various methods are available for binding, separating or analyzing the target substances mentioned above based upon complex formation between the substance of interest and another substance to which the target substance specifically binds. Separation of the resulting complexes from solution or from unbound material may be accomplished gravitationally, e.g. by settling, or, alternatively, by centrifugation of finely divided particles or beads coupled to the target substance. Accordingly, target substances can be bound with a complex that alters their density so that they can be separated by gravitational or centrifugal forces. More commonly, magnetic particles are used to bind target substances enabling them to be collected in a magnetic gradient.

Magnetic particles are well known in the art, as is their use in immune and other bio-specific affinity reactions. See, for example, U.S. Pat. No. 4,554,088 and Immunoassays for Clinical Chemistry, pp. 147-162, Hunter et al. eds., Churchill Livingston, Edinburgh (1983). Generally, any material which facilitates magnetic or gravitational separation may be employed for this purpose. However, processes relying on magnetic principles are preferred because high levels of recovery and purity are achievable by these methods, making them suitable for removal or isolation of rare cells from a mixed population of cells. Such separations include, but are not limited to, enrichment of CD34+ stem cells or immune cells from bone marrow or peripheral blood, isolation of fetal cells from maternal blood, isolation of transfected cells, and removal or isolation of tumor cells from various mixed cell populations. Separations may be accomplished by positive selection or negative depletion, or both, and cells recovered by such separation methods may be utilized for numerous purposes, including further analysis or therapeutic purposes (e.g., re-introduction of cell populations to patients).

Magnetic particles used for separation of biological materials generally fall into two broad categories. The first category includes particles that are permanently magnetizable, or ferromagnetic; and the second comprises particles that demonstrate bulk magnetic behavior only when subjected to a magnetic field. The latter are referred to as magnetically responsive particles. Materials displaying magnetically responsive behavior are sometimes described as superparamagnetic. However, materials exhibiting bulk ferromagnetic properties, e.g., magnetic iron oxide may be characterized as superparamagnetic when provided in crystals of about 30 nm or less in diameter. Larger crystals of ferromagnetic materials, by contrast, retain permanent magnet characteristics after exposure to a magnetic field and tend to aggregate thereafter due to strong particle-particle interaction.

Magnetic particles can be classified as large (about 1.5 to about 50 um), small (about 0.7-1.5 microns), and colloidal or nanoparticles (<200 nm). The latter are also called ferrofluids or ferrofluid-like particles and have many of the properties of classical ferrofluids. Liberti et al. pp. 777-790, E. Pelezzetti (Ed) “Fine Particle Science and Technology”, Kluwer Acad. Publishers, Netherlands. Small magnetic particles are quite useful in analyses involving bio-specific affinity reactions, as they are conveniently coated with bio-functional polymers (e.g., proteins), provide very high surface areas and give reasonable reaction kinetics. Magnetic particles ranging from 0.7-1.5 microns have been described in the patent literature, including, by way of example, U.S. Pat. Nos. 3,970,518; 4,018,886; 4,230,685; 4,267,234; 4,452,773; 4,554,088; and 4,659,678. Certain of these particles are disclosed to be useful solid supports for immunologic reagents. In addition to the small magnetic particles mentioned above, there is a class of large magnetic particles (>1.5 microns to about 50 microns) which also have superparamagnetic behavior. Such materials include those invented by Ugelstad (U.S. Pat. No. 4,654,267) and manufactured by Dynal (Oslo, Norway, now available through Invitrogen, part of Thermo Fisher Scientific). Polymer particles are synthesized, and through a process of particle swelling, magnetite crystals are embedded therein. Other materials in the same size range are prepared by performing the synthesis of the particle in the presence of dispersed magnetite crystals. This results in the trapping of magnetite crystals in a matrix-like material, thus making the material magnetic. In both cases, the resultant particles have superparamagnetic behavior, readily dispersing upon removal of the magnetic field. Unlike magnetic colloids or nanoparticles referred to above, such materials, as well as small magnetic particles, because of the mass of magnetic material per particle, are readily separated with simple laboratory magnets. Thus, separations are effected in gradients as low as a few hundred gauss/cm to about 1.5 kilogauss/cm.

On the other hand, based on theoretical calculations, colloidal magnetic particles (below approximately 200 nm) require substantially higher magnetic gradients—on the order of 100 kGauss/cm—for separation because of their diffusion energy, small magnetic mass/particle ratio and stoke drag. In spite of that, Liberti (unpublished results) discovered they can be separated in fields as low as 7-10 kGauss/cm. Based on this observation, the materials are believed to form nanoparticle magnetic chains in magnetic fields which dramatically alter their mass, thus making the theoretical calculations have little meaning.

U.S. Pat. No. 4,795,698 to Owen et al. relates to polymer coated, sub-micron size colloidal superparamagnetic particles. The '698 patent describes the manufacture of such particles by precipitation of a magnetic species in the presence of a bio-functional polymer. The structure of the resulting particles, referred to herein as single-shot particles, has been found to be a micro-agglomerate in which one or more ferromagnetic crystallites having a diameter of about 5-10 nm are embedded within a polymer body having a diameter on the order of 50 nm. These particles exhibit an appreciable tendency to remain dispersed in aqueous suspensions for observation periods as long as several months. Molday (U.S. Pat. No. 4,452,773) describes a material which is similar in properties to those described in the '698 patent of Owen et al. produced by forming magnetite and other iron oxides from Fe³⁺/Fe²⁺ via base addition in the presence of very high concentrations of dextran. Materials produced in this manner have colloidal properties. This process has been commercialized by Miltenyi Biotec GmbH (Bergisch Gladbach, Germany). Those products have proved to be very useful in cell separation assays.

Another method for producing superparamagnetic colloidal particles, also referred to as ferrofluids, is described by Liberti et al. in U.S. Pat. Nos. 5,597,531 and 6,551,843. In contrast to the particles described in the '698 patent, these latter particles are produced by directly coating a bio-functional polymer onto pre-formed superparamagnetic crystals which have been dispersed by sonic energy into quasi-stable crystalline clusters, ranging in size from about 25 to 120 nm. The resulting particles, referred to herein as direct-coated or DC particles, exhibit a significantly larger magnetic moment than the nanoparticles of Owen et al. or Molday et al., while having about the same overall size.

Magnetic separation techniques utilize magnetic field generating means to separate ferromagnetic bodies from a fluid medium. In contrast, the tendency of colloidal superparamagnetic particles to remain in suspension, in conjunction with their relatively weak magnetic responsiveness, requires the use of high-gradient magnetic separation (HGMS) techniques in order to separate such particles from a fluid medium in which they are suspended. In HGMS systems, the gradient of the magnetic field, i.e., the spatial derivative exerts a greater influence upon the behavior of the suspended particles than is exerted by the strength of the field at a given point. HGMS is useful for separating a wide variety of biological materials, including eukaryotic and prokaryotic cells, viruses, nucleic acids, proteins, and carbohydrates.

In methods known heretofore, biological material has been separable by means of HGMS if it possesses at least one determinant capable of being specifically recognized by and bound to a targeting agent, such as an antibody, antibody fragment, specific binding protein (e.g., protein A, protein G, streptavidin), lectin, and the like.

HGMS systems can be divided into two broad categories. One such category includes magnetic separation systems that employ a magnetic circuit that is situated externally to a separation chamber or vessel wherein the magnetic gradient is created by pole piece placement and design. Examples of such external separators (or open field gradient separators) are described in U.S. Pat. No. 5,186,827. In several of the embodiments described in the '827 patent, the requisite magnetic field gradient is produced by positioning permanent magnets around the periphery of a non-magnetic container such that the like poles of the magnets are in a field-opposing configuration. The extent of the magnetic field gradient within the test medium that may be obtained in such a system is limited by the strength of the magnets and the separation distance between the magnets. Hence, there is a finite limit to gradients that can be obtained with an external gradient system.

Another type of HGMS separator utilizes a ferromagnetic collection structure that is disposed within the test medium in order to 1) intensify an applied magnetic field and 2) produce a magnetic field gradient within the test medium. In one known type of internal HGMS system, fine wires, e.g., steel wool or gauze, are packed within a column that is situated adjacent to a magnet or within a magnetic dipole. The applied magnetic field, operating according to well-known principles of physics, creates very high gradients extending from the wire surfaces so that suspended magnetic particles will be attracted toward, and adhere thereto. The gradient produced on such wires is inversely proportional to the wire diameter, whereas magnetic “reach” decreases with diameter. Hence, very high gradients can be generated. One drawback of such internal gradient systems is that the use of steel wool, gauze material, steel microbeads or the like, may entrap non-magnetic components of the test medium by capillary action in the vicinity of intersecting wires or within interstices between intersecting wires or adjacent microbeads. Various coating procedures have been applied to such internal gradient columns (U.S. Pat. Nos. 4,375,407 & 5,693,539), however, the large surface area in such systems still makes recovery problematic due to adsorption. Hence, internal gradient systems are not desirable where recovery of very low frequency captured target entities is the goal of the separation. Further, automation of such separation systems is both difficult and costly.

In contrast, cell separations using HGMS based approaches with external gradients provide a number of conveniences. First, simple laboratory tubes such as test tubes, centrifuge tubes, or even vacutainers (used for blood collection) may be employed. When external gradients are of the kind where separated cells can, in principle, effectively be monolayered, as is the case with appropriately designed quadrupole/hexapole devices as described in U.S. Pat. No. 5,186,827 or the opposing dipole arrangement described in U.S. Pat. No. 5,466,574, washing of cells and subsequent manipulations are facilitated. Furthermore, recovery of the cells from tubes or similar containers is a simple and efficient process.

There are a number of important variables that affect the efficiency with which magnetic separations can be done as well as the recovery and purity of magnetically labeled cells. These include such considerations as: the number of cells being separated, the density of targeted determinants present on such cells, the magnetic loading per cell, the non-specific binding tendency of the magnetic material (NSB), the methodology employed for magnetically loading cells, the shape of the vessel, the composition of the vessel surface, and viscosity of the medium. Accordingly, optimization of magnetic separations of cells is not an easy or trivial task.

Of the above, a very important consideration in cell separation is, indeed, the methodology employed in the magnetic labeling step. There are two options for doing that: a direct or indirect approach. In the case of a direct labeling approach, typically a monoclonal antibody (mAb) or some other member of a specific binding pair is directly coupled to a magnetic nanoparticle, which is subsequently incubated with a cell mixture. Either by mixing in the case of large magnetic particles or by diffusion in the case of ferrofluid-like materials, magnetic particles specifically attach themselves to target cells. For large magnetic particles, such as Dynabeads, such reactions typically require 30 minutes to achieve sufficient magnetic loading. For colloidal magnetic nanoparticles or ferrofluids, such reactions require 10-20 minutes.

Alternatively, targets can be magnetically labeled via the indirect method which involves at least two steps. The first step is the incubation of cells with mAb or mAb coupled to some small molecule, such as biotin. That is followed by a second step which involves an incubation of the mAb labeled cells with a common capture magnetic particle, e.g. a goat anti-mouse magnetic particle or a streptavidin magnetic particle. If the first incubation step is performed at mAb concentrations of 1.0 ug/mL with cell concentrations of 1.0×10⁶ to 10⁸ cells per mL, it is well established that mAb labeling plateaus at about 20 minutes. On the other hand, if a mAb concentration of 5.0 ug/mL is used with similar cell concentrations, sufficient labeling is achieved in 5 minutes. In either case, it is a generally accepted practice to remove unbound mAb via centrifugation (known as “washout”) from the cell incubation mixture before addition of common capture magnetic material so as to prevent the latter from crosslinking with free mAb or free biotinylated mAb.

In the case of large magnetic particles, such as Dynabeads, a mAb ‘washout’ is required in order to prevent large and interfering aggregates of particle-mAb-particle from forming. Such aggregates not only negatively impact the separation but also trap target cells in large agglomerated clusters. On the other hand, with colloidal magnetic materials such as those disclosed in Liberti '531 and '843, it was discovered previously that unbound mAb removal is not required providing mAb labeling concentrations are not excessive (Liberti et al., U.S. Pat. No. 5,186,827). Not having to washout unbound mAb is a very significant advantage as it avoids mAb removal processes that are difficult to automate and require substantial time to perform. Additionally, it makes the indirect method significantly more attractive than the direct method in the versatility it affords as only one magnetic conjugate needs to be synthesized or manufactured, i.e., the common capture nanomagnetic material. A spectrum of cell separation kits using those principles is commercially available from STEMCELL Technologies, Inc. (Vancouver, BC).

In spite of the advantages of performing an indirect separation without having to remove excess mAb, the process does involve two incubations, which in most cases are a 10-15 minute mAb incubation (more typically 15 minutes), followed by a 10-15 minute magnetic labeling process. In the case of magnetic nanoparticles in the colloidal size range, where the no washout approach appears to work best, magnetic separations done in open field gradient separators require an additional 10-15 minute time period to effect separation. Accordingly, targeted cells are subjected to processes that require minimally 30 minutes and more typically 40 minutes. Even when the entire magnetic cell separation process is done at 0° C., cells can be damaged by prolonged processing. Additionally, the economic benefits that would accrue for shortening the processing time are quite obvious in regards to throughput for manual or automated processes. The ability to effect more cellular experiments with higher cell viability and higher throughput in a shorter time has vast economic and scientific value to researchers, scientists, technician, laboratories, universities and institutes worldwide and is easily calculable—affecting a panoply of scientific fields from: oncology, hematology, genomics and the broad spectrum of cell-based biological sciences.

One approach to shortening the time to separation, as well as, improving efficiency for magnetic separations has been to enhance magnetic loading in the case of cells or magnetic-target interactions in the case of macromolecular targets. One means for enhancing magnetic loading onto targets, when using colloidal magnetic nanoparticles that substantially improves the efficiency of magnetic separations, employs a scheme that causes unbound magnetic nanoparticles to aggregate onto cell bound nanoparticles. It is referred to as ‘controlled aggregation’ (U.S. Pat. No. 6,623,982). This enhanced magnetic loading is accomplished by constructing a nanoparticle that had coupled to its surface two receptors, e.g., streptavidin and a mAb specific for target cells. After sufficient incubation to allow the nanoparticle to bind to target cells via the mAb reaction, a second component containing two or more biotins, in this example, is added to cause free nanoparticles to bind to cell bound nanoparticles via the biotin-streptavidin reaction. By this process, cells bearing low-density receptors are made significantly more magnetic and readily captured with open-field gradient devices. In the case where the added ‘crosslinking’ reagent is bi- or multivalent des-biotin, aggregation can be readily reversed by the addition of biotin based on the extraordinary difference in their affinity for streptavidin. Thus, a system is created where magnetic loading is used to capture cells that might not be retrieved, but reversed so that cell interrogation can be done without being obscured by the presence of excessive magnetic nanoparticles.

Another means for creating greater magnetic loading onto targets is to cause magnetic particles to move relative to cells or vice versa. This has been accomplished by Kreuwel et al. (U.S. Pat. No. 6,764,859), by means of devices that use magnetic gradients brought into proximity of vessels containing magnetic particles which cause movement of such particles through solutions, thus creating collisions that result in magnetic loading of targets. Terstappen et al. (U.S. Pat. No. 6,551,843) disclose methods for magnetic loading onto target cells by either centrifuging cells through mAb specific colloidal magnetic materials (ferrofluids) or by placing such mixtures into quadrupole magnetic devices that cause ferrofluids to move radially to the walls of the vessel. For the centrifugally enhanced magnetic loading process, it is important to note that ferrofluids require very high g-forces to pellet and accordingly cells readily centrifuge through them. In the quadrupole examples cited, movement of ferrofluid through cell mixtures required about 15 minutes to complete.

There are several drawbacks in using quadrupole separators for creating magnetic loading onto target entities, the first of which is the long time component required to accomplish such loading. Moreover, as the ferrofluid moves through the mixture there will be regions wherein there are target cells but little or no magnetic materials, as the latter has moved to the walls of the vessel. Thus the relative motion that creates cell-magnetic particle interaction also results in regions where target cells are not in proximity of magnetic nanoparticles. In examples, given below, we show there is a substantially better way to accomplish relative motion of targets and magnetic nanoparticles that result in enhanced magnetic loading relatively quickly.

### SUMMARY OF THE INVENTION

From extensive experiments using much improved ferrofluid-like materials (70 to about 280 nm magnetic colloids) to facilitate magnetic based cell separations, we have frequently found that an indirect magnetic labeling scheme results in higher yield and purity of isolated cells as compared to a direct labeling method. However, the need to generally perform two incubations (target labeling and common capture labeling) and two centrifugations (for effective removal of target mAb) makes this approach somewhat more difficult, time consuming and automation unfriendly than the direct procedure where mAb or targeting component is directly coupled to a magnetic particle. We have also found with mAb pre-labeled cells that the magnetic labeling step is maximized at 20 minutes incubation when done at room temperature even though 10 minutes will suffice in many instances, depending on the desired yield. At 0° C., that reaction takes even longer. To achieve maximum magnetic labeling at minimal input of common capture ferrofluid (desirable because of the potential negative effects of excess magnetic material inducing non-specific binding) in a reduced time period, we have generated and optimized a magnet/mixing scheme that reduces the 20 minute room temperature incubation period to 5 minutes, even when done at 0° C.

We have further surprisingly discovered that by lowering the concentration of mAb typically used to pre-label cells in an indirect labeling scheme, as well as, minimizing the numbers of determinants on that mAb to which common capture ferrofluid is directed that all the components (cells, labeling mAb and common capture ferrofluid) can be mixed, incubated simultaneously and subsequently magnetically separated to give results equivalent to a 2-step indirect procedure with mAb washout. In other words, we have discovered that multiple labeling reactions (mAb to cell determinants, common capture particle to free mAb, common capture particle to cell bound mAb) can be done substantially simultaneously and result in high yield and high purity of targeted bioentities. Also, if a magnetic/mixing scheme is applied to these simultaneously reacting components, maximum labeling, as determined by the ability to magnetically separate such mixtures, can typically be achieved in 5 minutes at 0° C. Thus we disclose here means for reducing the time for a process that normally takes 25-30 minutes to just 5 minutes.

These discoveries make it possible to effectively convert in situ any indirect immuno-magnetic cell separation to the equivalent direct process. It clearly obviates, in likely all instances, the need to prepare direct mAb-magnetic nanoparticle conjugates for performing such separations. Using this approach, appropriate common capture reagents that are directed to small molecules such as biotin or to limited numbers of antigen determinants on targeting mAbs can be prepared. In the case of common capture reagents directed to cell labeling antibodies, it would be evident that targeting those agents to limited numbers of determinants on the Fc region is desirable. It should also be clear that common capture nanoparticles need not be magnetic and will be useful in a variety of other applications.

These discoveries, as well as variables within the system that can be adjusted and tailored for specific applications, will make automation of cell separations simpler, require less time, reduce cost and provide isolated cells upon which fewer manipulations will have been performed as compared to prior art systems.

There are other applications where it would be advantageous to form direct conjugates in situ via the simultaneous addition of appropriate reagents as disclosed herein. One important example is the work of June et al., U.S. Pat. No. 8,637,307, who demonstrated that engineered cells bearing anti-CD3, or anti-CD2, as well as anti-CD28 when incubated with T cells very effectively led to polyclonal T-cell proliferation—a very important event for immunotherapy, as June and colleagues have so elegantly demonstrated. U.S. Application No. 2014/0087462 of Sheffold and Assenmacher reviews the evolution of the invention of the '307 patent for causing polyclonal T-cell proliferation via the aforementioned stimulators when they are attached to flat and spherical surfaces, where the latter are of various diameters and surface properties. They further demonstrate that nanoparticles that result from the Molday process, U.S. Pat. No. 4,452,773, are very capable of acting as T-cell proliferators when either or both CD3 and CD28 are attached to single iron-dextran microspheres or where appropriate mixtures of microspheres that bear the respective antibodies are used. A more straightforward, more economical and more versatile means for accomplishing such stimulations would be to use the invention described herein, where anti-CD3 and anti-CD28 and an appropriate common capture agent are simultaneously added to T-cells. In that way, ratios of anti-CD3/anti-CD28 as well as any other relevant monoclonals could be used, thus allowing the requisite T-cell receptor reactions and clustering to take place in situ. For example, if anti-CDs of two different isotypes are employed for stimulation and if two different common capture agents are used where each has a different specificity, e.g., anti-isotype 1 and anti-isotype 2, then the potential advantages of stimulation by two different nanoparticle-mAb entities can readily be determined.

In accordance with one aspect, the present invention provides an efficient and effective method of forming a bioconjugate. The method involves the following steps: (i) combining substantially simultaneously in a biologically compatible medium (a) a target bioentity having at least one characteristic determinant, (b) at least one targeting agent, each targeting agent comprising multiple binding units, each binding unit having at least one binding site and at least one recognition site, the targeting agent being effective to bind specifically through at least one of the binding sites to at least one determinant of the target bioentity to yield a labeled bioentity, and (c) a nanoparticle-borne capture agent having at least one binding moiety that binds specifically to at least one of the recognition sites of the labeled bioentity, thereby forming the bioconjugate, and subjecting the medium including the target bioentity, targeting agent and capture agent to incubation conditions of temperature (about 0-44° C.) and time (3-30 minutes) to promote formation of the bioconjugate. The nanoparticle-borne capture agent used in practicing this invention has a physical property rendering the formed bioconjugate differentiable in the medium. Furthermore, each binding unit of the targeting agent(s) has about 1-10 recognition sites present thereon, and each capture agent has about 1,000 to 8,000 binding moieties per nanoparticle, with the number of binding moieties on the capture agent being at least two fold greater than the average number of recognition sites on all binding units of the targeting agent present in the medium.

In accordance with another aspect, the present invention provides a method of forming a stimulated T cell bioconjugate by combining substantially simultaneously in a biologically compatible medium, CD3⁺ cells (T cells), an anti-CD3 antibody, an anti-CD28 antibody and a magnetic nanoparticle-bound anti-Fc antibody, the concentration of anti-CD3 antibody and anti-CD28 antibody in the medium being in the range of about 0.05 to about 1.5 μg/ml, and the concentration of magnetic nanoparticle-bound anti-Fc antibody in the medium being equal to or greater than the anti-CD3 antibody concentration. Thereafter, the medium including the CD3+ cells, anti-CD3 antibody, anti-CD28 antibody, and magnetic nanoparticle-bound Fc antibody is incubated under conditions of temperature (10-42° C.) and time (5-30 minutes) to promote formation of the stimulated T cell bioconjugate. Then, the medium is exposed to a magnetic field gradient to form a stimulated T cell bioconjugate aggregate. Next, exposure of the medium to the magnetic field gradient is terminated and the stimulated T cell bioconjugate aggregate is dispersed in the medium.

In a further aspect, the present invention provides a method of separating a subpopulation of cells of interest having at least one characteristic determinant from a mixed cell population. The method comprises combining substantially simultaneously in a biologically compatible medium, the mixed cell population, at least one targeting agent, each targeting agent comprising multiple binding units, each binding unit having at least one binding site and at least recognition site, the targeting agent being effective to bind specifically through at least one of the binding sites to at least one characteristic determinate of the subpopulation of cells, thereby yielding a labeled cell, and a nanoparticle-borne capture agent having at least one binding moiety that binds specifically to at least one recognition site, thereby forming a bioconjugate comprising the subpopulation of cells, with the combination being contained in a non-magnetic containment vessel having a wall surface in contact with the medium, and subjecting the medium including the mixed cell population, targeting agent, and capture agent to incubation conditions of temperature (about 0-37° C.) and time (3-30 minutes) to promote formation of the bioconjugate. Each binding unit of the targeting agent(s) has about 1-10 recognition sites present thereon, and each capture agent has about 1,000 to 8,000 binding moieties per nanoparticle, with the number of binding moieties on the capture agent being at least two fold greater than the average number of recognition sites on all binding units of the targeting agent in the medium.

### DETAILED DESCRIPTION OF THE INVENTION

The invention will now be further described in reference to certain preferred embodiments in the following description:

During the course of improving immuno-magnetic cell separations, we have encountered some very surprising results as regards what are commonly referred to as ‘indirect’ immuno-magnetic separations. By an indirect process, we refer to procedures where target cells are first incubated with a targeting agent, typically a monoclonal antibody, and where that labeling step is followed by a second step that links common capture agent to the targeting agent, in our case a magnetic nanoparticle bearing an appropriate molecule that binds to such labels. A cell so labeled by this ‘indirect’ or two-step process can readily be retrieved by exposing that cell to an appropriate magnetic gradient. The advantages of such indirect processes are well known. They are particularly advantageous as one common capture agent functions with a spectrum of appropriate targeting agents, again typically monoclonal antibodies, for performing any desired separation. Besides the obvious economic advantages of only having to prepare or manufacture one kind of magnetic nanoparticle, the common capture agent, not having to prepare monoclonal nanoparticle conjugates is a substantial advantage as that is a costly, time consuming process, is an inefficient and wasteful use of monoclonals and clearly storing and dispensing monoclonals as opposed to nanoparticle conjugates is advantageous. Accordingly, ways in which indirect immuno-magnetic separations, or any other indirect labeling process, can be improved can have immense scientific, clinical and economic significance.

In the performance of an indirect process, monoclonal antibodies are typically incubated with a cell mixture for periods of 15-20 minutes and usually at a level of mAb that is excessive to ensure that sufficient cell surface determinants are labeled so that adequate common capture magnetic materials can subsequently be attached to the cell resulting in efficient magnetic separation. In an ideal situation, clearly the objective is to attach to a target cell only that amount of magnetic common capture nanoparticle that will ensure separation of the target in the magnetic gradient used for cell retrieval. That makes good economic sense, helps preserve cell integrity and also facilitates subsequent manipulations of captured target cells such as interrogation, culturing, specific activation and the like. In addition, as much of the common capture particle should be attached to cells as possible so as to not collect target cells in a sea of non-cell bound capture agent. An overabundance of unbound capture antigen obscures cells and makes subsequent operations more difficult.

With those objectives in mind, as well as a desire to simplify the indirect cell separation process, we first examined magnetic loading onto cells and optimized that process for ferrofluid-like materials. Our goal was to minimize common capture input while maximizing cell loading for separations in open field magnetic gradient separators. Note that optimizations disclosed here are done with open field separators that have magnetic gradients limited to about 12-14 kGauss/cm. From extensive experience with 125-135 nm streptavidin ferrofluids [SAFF] prepared by improved methods disclosed in Liberti et al. '531 and '843, 8-10 ug of SAFF is typically used to perform separations on 1-2×10⁷ labeled cells. Making the assumptions that these materials have an approximate spherical crystalline core of magnetite that is 120 nm diameter and a protein/polymer coating that is about 10 nm, it can be calculated from iron and carbon analysis data that 1 ug of SAFF contains about 2×10⁸ nanoparticles. When 1×10⁷ cells are targeted, typically 10 ug of SAFF are used which amounts to 200 SAFF nanoparticles per cells. Even though that amount of SAFF hardly changes the appearance of the cell suspensions (SAFF is black), 200 nanoparticles per cell seems excessive. That value seems particularly excessive as with magnetic gradients 10 times greater cells can be captured with as little as 1-3 SAFF nanoparticles.

Table I tabulates the results of experiments on the kinetics of binding of SAFF to cells at RT. As can be seen, saturation does not occur to at least 20 minutes incubation. At 0° C., as would be expected, SAFF binding is even slower. As can be seen from the table that for the conditions used in these experiments, about 10% of target cells would be missed if only 10 minute ferrofluid incubation is employed.

TABLE I
Magnetic incubation time and the separation
of magnetically labeled cells at 23° C.

| Time of incubation | % Cells captured |
|---|---|
| 10 min | 90 |
| 15 min | 96 |
| 20 min | 99 |

Experimental conditions: 1.0 mL cells (10⁸ cells/mL) labeled with 1 ug/mL biotin-anti-CD3 mAb for 10 min with mAb washout followed by addition of 50 ug SAFF in 15 uL, incubated for 10 min, diluted 10-fold to a concentration of 10⁷ cells/mL and separated in a quadrupole for 10 min. Note that a higher concentration of SAFF would need to be employed to get higher yields at incubations of around 10 minutes.

As already noted, various protocols of magnetic ‘incubation’ for enhancing SAFF loading have, indeed, been employed. For the protocols used in Terstappen et al. '843 and Kreuwel et al. '859, there are some inherent limitations that should be noted. In the '843 disclosure, magnetic enhanced binding was done in quadrupole separators that pull ferrofluids radially. There are two negative consequences of doing that. First, in quadrupoles wherein radial separation takes place just from geometric considerations, magnetic materials dilute as they move outward, thus leaving cells towards the center deficient in magnetic nanoparticles which can only have negative consequences vis-à-vis cell labeling. Secondly, the lengthy periods in which the radial fields were used for incubation leaves a significant number of cells deficient in surrounding magnetic labeling. In Kreuwel '859, pulling magnetic materials from side to side of a vessel will also leave cells on the side opposite the magnetic gradient deficient in labeling materials.

To obviate the above mentioned issues, we devised an improved scheme for performing magnetic loading enhancement. This was done by manipulating test tubes containing SAFF and biotin-mAb labeled cells (i.e., cells pre-labeled with a biotin-mAb and washed out) in the gradient fields of a yoked magnet containing north-south pole neodymium-iron-boron [NdFeB] block (0.5″×0.5″×2.0″) magnets having a gap of 3.0″. Manipulations were done by moving sample tubes from pole face to pole face, without turning the tubes, i.e. re-orienting them, such that ferrofluid is swept from side to side within the tube as the tube is moved from pole to pole. The dwell time (30, 45, 60 and 90 seconds) during which tubes were adjacent to a pole and the level of SAFF (2, 4, 5 and 6 ug/10⁷ cells) were varied to find the condition where about 50% of the cells could subsequently separate in a quadrupole device. Total incubation times for these experiments were 5 minutes. From these experiments, it was determined that 44% of cells (10⁷ input) could be separated when incubated with 2 ug SAFF with a dwell time of 45 seconds. Using a dwell time of 40 sec, total times of 320 sec (4 N-S cycles) and SAFF at a level of 2 ug per 10⁷ targeted cells, various additional manipulations were done to see if the % of cells recoverable by subsequent magnetic separation could be increased. Table II shows the results of these experiments.

TABLE II
Effects of various magnetic and non-magnetic
manipulations on SAFF binding to cells¹

| | % of cells |
|---|---|
| Condition | separated |
| Alternating poles, dwell time 40 sec, vortex in between | 70 |
| No magnetic field, vortex tube every 40 sec | 51 |
| Constant vortexing of tube with magnet affixed to one side | 32 |
| Control tube - untouched on lab bench away from any fields | 38 |

¹Cells at 10⁷ per mL, SAFF at 2 ug/mL; total processing time of 320 seconds

From Table II it is clear that for magnetic nanoparticles of the size and magnetic susceptibility of those used here (typical of colloidal materials currently used with open field separators) that magnetic incubation done by creating movement of the nanoparticles from side to side of a vessel with a pole face dwell time of about 40 seconds (at which there is no visually apparent ferrofluid collection on the sides of tubes after a magnetic sweep) and very importantly with vortexing in between to redistribute unbound ferrofluid after each ‘sweep’, that very significant enhancement of magnetic labeling can be achieved as evidenced by the ability to separate such labeled cells (70% vs 38%). For larger magnetic particles or those that might have greater magnetic susceptibility, it would be easy to find appropriate magnetic sweeping forces, dwell times and mixings to achieve enhanced loading based on this approach. It is very noteworthy for the control tube where no manipulations are done or fields applied that only about half the cells are separated—showing how effective this method is. It is important to note that the dwell time in a particular magnetic gradient that causes ferrofluids to move through a solution will be greatly dependent on the strength of the gradient, the gradient shape, the temperature and viscosity of the mixture as well as the cell concentration. Accordingly, it would be clear that dwell time for a particular magnetic arrangement and test system would best be determined experimentally.

Capturing 70% of target cells with this methodology and using just 2 ug of SAFF per 10⁷ target cells, equates to an input of about 60 nanoparticles per cell. If only one SAFF particle binds to one mAb, these results suggest that very little mAb binding is required. On the other hand, SAFF could be binding to multiple mAbs. Nonetheless, these results raise the question: how much mAb labeling is actually required to achieve efficient separation with an indirect labeling process (2-step) as well as a second question which is: if the mAb primary labeling concentration can be reduced significantly so as not to overwhelm the capacity of the common capture agent, and if there are a limited number of determinants on the mAb that the common capture agent binds to, then can the indirect method be done by mixing all components together in the same ‘pot’? In other words, can a cell mixture containing target cells be mixed with an appropriate mAb, with limited target binding sites for a common capture agent, and the common capture agent substantially simultaneously? And, would the kinetics of all three reactions (mAb binding to target cells, SAFF binding to free mAb, and SAFF binding to cell-bound mAb) taking place simultaneously be favorable or somehow enhanced so that incubation times are reduced and the quality of separations is improved? Or, alternatively, would the potential of common capture clustering with multivalent mAb, e.g. SAFF-mAb-SAFF, prove to be a negative aspect of a one-pot, simulataneous reaction?

We have discovered that, provided the number of binding sites on the targeting component (e.g., mAb) available for common capture agent binding is reasonably low that a highly efficient indirect immuno-magnetic labeling or separation procedure can be done by mixing all the components simultaneously. We have further discovered that labeling reaction kinetics are surprisingly favorable when done without any added magnetic manipulations and that they are even more favorable when a magnetic manipulation procedure as described above is performed on the reaction mixture. In examples, shown below, it is demonstrated that such simultaneous reactions are sufficiently completed, as assessed by subsequent magnetic separation of targets, in about 5 minutes compared to a process involving a 15 minute mAb incubation step followed by a 10-15 minute common capture particle incubation in the case where there is no mAb washout—a total processing time of 25-30 minutes. Further it has been discovered that the order of addition of the reacting components does not appear to be important, i.e. mAb combined with cells followed by common capture agent or cells combined with common capture agent followed by mAb addition. In the latter case, as no reaction occurs when cells and common capture are mixed, there will be occasions where this approach is preferred as good mixing can be achieved prior to any reaction that will occur once mAb is added. However, based on the volumes of components used in these studies, it has been found advantageous to first place labeling mAb (typically 5-20 ul) into a vessel, followed by rapid addition of cells with immediate mixing, and then to add common capture nanoparticles and mix.

Reactions can also be performed where mAb and cells are mixed and the common capture nanoparticles are added after 30-60 seconds. It is noted that the common capture agent or nanoparticles need not be magnetic in the case of other applications where it is desirable to label target cells for some other process. Hereafter, we refer to the process for doing an indirect assay protocol by mixing all components together and promoting simultaneous reactions via magnetic manipulations as simultaneous labeling and magnetic mixing (SLAMM). In order to additionally promote labeling and conserve reagents, SLAMM can be done advantageously at cell concentrations 5 to 10 times higher than the cell concentration used during magnetic separation. Clearly the higher the concentrations of the reactants the more rapidly such reactions occur.

However, the option of increasing the concentration of reactants will depend on the desired outcome and parameters of the separation mixture. We describe SLAMM separations in which incubation and separation are done at the same cell concentrations hereafter as SLAMM. Alternatively, such separations where the sample is diluted prior to separation either 5-fold or 10-fold, for example, are referred to as SLAMM-5 and SLAMM-10, respectively. The examples provided demonstrate the performance of the SLAMM protocol and important parameters of this protocol.

The following definitions will facilitate the understanding of the methods used in accordance with the present invention:

The term “substantially simultaneously” as used herein in reference to the combining of a target bioentity, targeting agent(s), and suitable capture agent(s), refers to an approach wherein these components are combined in a biologically compatible medium at the same time, or nearly the same time, in such a manner that allows for in situ formation of bioconjugates. This includes an approach where a targeting agent is added to medium containing a target bioentity and capture agent in order to initiate a reaction. The formation of bioconjugates in situ by substantially simultaneously combining the required components may also be achieved by adding a capture agent to a medium containing a target agent and target bioentity within a short period of time, on the order of 5 minutes or less, preferably less than 1 minute, after combining a target agent and a target bioentity, thus promoting the formation of bioconjugates and avoiding potential crosslinking or agglomeration of a target agent and capture agent.

The term “biologically compatible medium” as used herein refers to a liquid in which the target bioentity, and other agents used in practicing this invention, is/are maintained in an active form or viable state. A preferred biologically compatible composition is an aqueous solution that is buffered using, e.g. Tris, phosphate or HEPES buffer, containing salt ions. Usually the concentration of salt ions will be similar to physiological levels. Biologically compatible media may include stabilizing agents and preservatives.

The term “capture agent” is made up of “capture agent units” and as used herein refers to a material that has affixed to it a protein, or some other molecule, with binding sites that are capable of forming specific binding pairs with a particular molecular recognition site common to some class of molecules (typically macromolecules) or specific molecules that can be coupled to some class of macromolecule. Binding pairs that are commonly used for such applications include avidin-biotin, streptavidin-avidin, species 1 anti-species 2 Fc-species 2 antibody (where the species 2 antibody is generally referred to as the targeting or primary antibody and the species 1 antibody is referred to as the secondary antibody, e.g. the specific binding pair consisting of a goat anti-mouse Fc and a mouse mAb, which are the secondary antibody and primary antibody, respectively). Capture agents typically include solid supports of various sizes and properties to which molecules with binding sites can be affixed. The use of macromolecules like avidin, streptavidin, neutravidin, and secondary antibodies bound to a surface such as micro-titer well that will form binding pairs with targeting agents is well known in the art. Similarly, the attachment of these macromolecules to particles, making them common capture agents, is also well known in the art.

The term “target bioentity” as used herein refers to a variety of materials of biological or medical interest, including eukaryotic and prokaryotic cells, subcellular organelles, viruses, proteins, nucleic acids, carbohydrates, ligands or complex molecules comprising nucleic acids, proteins, lipids and carbohydrates. A biological material is separable by the methods described herein if the material possesses at least one determinant, which is capable of being recognized by and bound to a receptor or ligand. If the target bioentity is a cell, it is also referred to herein as a “target cell.”

The term “characteristic determinant” as used herein in reference to a target bioentity, refers to that portion of the target bioentity which may be specifically bound by a targeting agent and is involved in, and responsible for, selective binding to the target bioentity. In fundamental terms, determinants are molecular contact regions on a target bioentity that are recognized by receptors or targeting agents and include substances such as antigens, haptens, and other complex molecules (e.g., carbohydrates, glycoproteins, etc.).

The term “targeting agent” as used herein refers to any substances or group of substances having a specific binding affinity for a given characteristic determinant of a target bioentity, to the substantial exclusion of other substances. Monoclonal antibodies are preferred for use as a targeting agent; however, other targeting agents include polyclonal antibodies, antibody fragments, non-antibody receptors, ligands, streptavidin or avidin-labeled reagents, hapten-labeled reagents, and fluorescently-labeled antibodies (e.g., phycoerythrin or fluorescein isothiocyanate conjugates).

The term “specific binding pair” as used herein describes a pair of molecules which have particular specificity for each other and which in normal conditions bind to each other in preference to binding to other molecules. Examples of specific binding pairs are antibodies and their cognate epitopes/antigens, ligands (such as hormones, etc.) and receptors, avidin/streptavidin and biotin, lectins and carbohydrates, and complementary nucleotide sequences. Various other determinant-specific binding substance combinations are contemplated for use in practicing the methods of this invention and will be apparent to those skilled in the art. In the context of the present invention, a specific binding pair can be formed between the “binding site” of a molecule associated with a capture agent and the “recognition site” of a corresponding target antigen.

The term “magnetically responsive material” is used herein to refer to particles that are permanently magnetized and particles that become magnetic only when subjected to a magnetic field. The latter are referred to herein as “magnetically responsive particles.” Materials displaying magnetically responsive behavior are sometimes described as superparamagnetic. However, certain ferromagnetic materials, e.g., magnetic iron oxide, may be characterized as magnetically responsive when the crystal size is about 30 nm or less in diameter. Larger crystals of ferromagnetic materials, by contrast, retain permanent magnet characteristics after exposure to a magnetic field and tend to aggregate thereafter. Magnetically responsive colloidal magnetite is known. See U.S. Pat. No. 4,795,698 to Owen et al., which relates to polymer-coated, sub-micron size magnetite particles that behave as true colloids.

The term “antibody” as used herein, includes immunoglobulins, monoclonal or polyclonal antibodies, immunoreactive immunoglobulin fragments, chimeric antibodies, haptens and antibody fragments, and molecules which are antibody equivalents in that they specifically bind to an epitope on the antigen of interest (e.g. the TCR/CD3 complex or CD28). An antibody may be primatized (e.g., humanized), murine, mouse-human, mouse-primate, or chimeric and may be an intact molecule, a fragment thereof (such as scFv, Fv, Fd, Fab, Fab′ and F(ab)′₂ fragments), or multimers or aggregates of intact molecules and/or fragments. An antibody may occur in nature or be produced, e.g., by immunization, synthesis or genetic engineering. Preferred antibody fragments for use in T cell expansion are those which are capable of crosslinking their target antigen, e.g., bivalent fragments such as F(ab)′₂ fragments. Alternatively, an antibody fragment which does not itself crosslink its target antigen (e.g., a Fab fragment) can be used in conjunction with a secondary antibody which serves to crosslink the antibody fragment, thereby crosslinking the target antigen. A number of anti-human CD3 monoclonal antibodies are commercially available, exemplary are OKT3, prepared from hybridoma cells obtained from the American Type Culture Collection, and the monoclonal antibody G19-4.

The term “stimulated” and “activated” are used herein to refer to the state of a cell following sufficient cell surface moiety ligation to induce a noticeable biochemical or morphological change. For example, within the context of T cells, such activation refers to a cell that has been sufficiently stimulated to induce cellular proliferation or to induce cytokine production.

The term “enrichment” as used herein refers to increasing the ratio of the target cells to total cells in a biological sample. In cases where peripheral blood is used as the starting materials, red cells are not counted when assessing the extent of enrichment. Using the method of the present invention, circulating epithelial cells (Allard et al, Clin Cancer Res Oct. 15, 2004, 10:6897) may be enriched relative to leukocytes to the extent of at least 2,500 fold, more preferably 5,000 fold, and most preferably 10,000 fold.

The following examples are provided to illustrate certain embodiments of the invention. They are not intended to limit the invention in any way.

### Example 1

### SLAMM-5 Results for T Cell Capture Using Anti-CD3 (+/−Biotin) and SAFF and/or Goat Anti-Mouse Fc Ferrofluid

CD3 mAb was obtained from Tonbo Biosciences (San Diego, Calif.). Two biotin labeled mAb conjugates were prepared using the biotin extender reagent from Setareh Biotech (Eugene, Oreg.) by well-known techniques and determined to have 0.7 and 4.0 biotins per mAb via the HABA/Avidin assay from Sigma (St. Louis, Mo.). A third biotinylated anti-CD3 mAb was obtained from Ancell Corporation (St. Paul, Minn.) that was of a 10:1 biotin/mAb ratio. The T-cell line HPB-MLT grown in culture was used as a source of target cells. Cells were harvested, tested for viability and resuspended in a proprietary neutral pH isotonic ferrofluid compatible buffer.

Two common capture nanoparticles were synthesized for these studies by coupling onto a 135 nm ferrofluid either goat anti-mouse Fc (obtained from Southern Biotech, Birmingham, Ala.) or streptavidin (ProZyme, Hayward, Calif.). Based on biotin binding capacity of the streptavidin ferrofluid [SAFF] and assuming that one streptavidin molecule can only bind one biotin-mAb, this SAFF preparation can bind greater than 1 mg mAb/mg ferrofluid.

From extensive preliminary experiments, it was found convenient to perform SLAMM reactions at cell concentrations of about 1-5×10⁷ target cells per mL. Furthermore, since the order of addition of components for the incubation reactions gave identical results, it was found convenient to add the components in an order that takes into consideration the component volume and best means to obtain or promote mixing. Accordingly, mAb or biotinylated mAb (smallest volume) was placed in a test tube (Eppendorf, 1.5 mL conical), a cell suspension was rapidly added and mixed well, and then a volume of the appropriate ferrofluid conjugate was added and mixed well. In some experiments, the mAb-cell mixture was allowed to stand 30 seconds before addition of ferrofluid conjugate; however, this approach had no apparent effect on the outcome. Immediately after all components were mixed, samples were subjected to cycles of being placed in a magnetic gradient and vortexing in the absence of any field gradient. Accordingly, the tubes were placed against the pole face of small block (0.5″×0.5″×0.5″) NdFeB rare-earth magnets (N52 grade) for 40 seconds, immediately removed and vortex mixed, and then replaced against the pole face. These cycles were repeated for total times of 5, 7 or 10 minutes at 40 second intervals. At the completion of SLAMM, samples were immediately diluted 5-fold with buffer, mixed, and placed in quadrupole separators for 10 minutes. For quantitation, supernatants were recovered and cells remaining uncollected were counted in a Countess Automatic cell counter (Life Technologies (Carlsbad, Calif.). Collected cells were typically examined microscopically. The results of these experiments are set forth in Table III.

TABLE III
Separation efficiencies for SLAMM-5 protocols for biotin mAb—streptavidin
ferrofluid (SAFF) and mouse antibody—goat anti-mouseFc ferrofluid (G@mFcFF) indirect
systems¹

| | | | | | | | | | 5 minute | 1 |
|---|---|---|---|---|---|---|---|---|---|---|
| | | | | | | | | | simultaneous | |
| | | | | | | | | % | incubation | |
| | SLAMM conditions = Simultaneous mixing and | | | | | Separation | | Cells | reactions— | |
| | labeling of all components and 40 second exposure to | | | | | Conditions (10 | | separated | not magnetic | |
| | magnetic gradient + 1 second vortex mixing cycles. | | | | | minutes in | | (SLAMM) | mixed | |
| | Time for | | | | | quadrupole | | % | % cells | 2 |
| | immuno- | | mAb type | | mass | device). | | Cells | separated | |
| | magnetic | mAb | biotin:Ig | ug | ratio of | mAb | FF | separated | (no magnetic | |
| | labeling | ug/mL | ratio | FF/mL | mAb/FF | ug/mL | ug/mL | (SLAMM) | mixing) | |
| Effect of | 5 min | 0.3 | 0.7:1 | 25 | 0.012 | 0.06 | 5 | 83% | 61% | 3 |
| biotin:Ig | 5 min | 0.3 | 4:1 | 25 | 0.012 | 0.06 | 5 | 78% | 46% | 4 |
| 0.7, 4, 10 | 5 min | 0.3 | 10:1 | 25 | 0.012 | 0.06 | 5 | 64% | 15% | 5 |
| 4 biotin/ | 5 min | 0.27 | 4:1 | 18.2 | 0.015 | 0.06 | 4 | 36% | | 6 |
| anti-CD3; | 10 min | 0.27 | 4:1 | 18.2 | 0.015 | 0.06 | 4 | 54% | | 7 |
| Effects of | 5 min | 0.27 | 4:1 | 22.7 | 0.012 | 0.06 | 5 | 82% | | 8 |
| [SAFF] | 10 min | 0.27 | 4:1 | 45.5 | 0.006 | 0.06 | 10 | 90% | | 9 |
| and | | | | | | | | | | |
| incubation | | | | | | | | | | |
| time | | | | | | | | | | |
| 0.7 biotin | 5 min | 0.30 | 0.7:1 | 25.0 | 0.012 | 0.06 | 5 | 83% | 66% | 10 |
| anti-CD3; | 5 min | 0.55 | 0.7:1 | 22.7 | 0.024 | 0.12 | 5 | 84% | | 11 |
| Effects of | 5 min | 0.55 | 0.7:1 | 45.5 | 0.012 | 0.12 | 10 | 95% | | 12 |
| [mAb] and | 5 min | 0.55 | 0.7:1 | 68.2 | 0.008 | 0.12 | 15 | 96% | | 13 |
| [SAFF] | | | | | | | | | | |
| G@mFcFF | 5 min | 0.55 | @CD3 | 22.7 | 0.024 | 0.12 | 5 | 94% | 80% | 14 |
| mag/vortex | | | mAb | | | | | | | |
| testing | | | | | | | | | | |

¹Cells were separated at 10⁷ cells/mL, and processed through SLAMM protocol at 5 × 10⁷ cells/mL

The top three lines of Table III show results of the effects of biotin valence of mAb used for labeling reactions in the SLAMM protocol. mAbs used for these experiments were biotinylated to 0.7, 4.0 and 10 biotins per antibody molecule. The data shows that the higher valence mAb generally gave lower efficiency of capture.

Given that the 10 biotin per mAb agent ‘consumes’ or occupies more streptavidin binding sites, two factors could be operative in lowering the efficiency of separation: (1) large clusters of mAb-streptavidin could be forming which may not subsequently bind to cells or (2) mAb may be lost in such clusters and not available to label cells. There may also be other explanations. For the 0.7 biotin conjugated mAb (row 3) recovery might be expected to be higher as there is likely no possibility of crosslinking; on the other hand, 30% bound mAb cannot be targeted by SAFF as they have no biotin. It seems likely that this could explain the lower separation efficiency. Row 10 is a repeat of the row 3 experiments with identical results. Rows 10 and 11 give about the same separation efficiency even though the mAb level is higher for the latter. However, rows 11, 12, and 13 show that at the higher mAb level when SAFF is increased from 5 ug to 10 ug and then to 15 ug, separation efficiencies rise above 90%. Cells not collected in the experiments using 10 and 15 ug SAFF were found not to be magnetic

From the above Table III, the magnetic capture efficiencies of biotin-mAb at biotin conjugations of 0.7, 4.0, and 10, are 83%, 78% and 64%, respectively. One possible explanation is that at the higher biotin/mAb ratios there is more opportunity for SAFF-mAb-SAFF crosslinking or agglomeration which results in complexes that merely consume mAb and thus limit amounts of mAb available for targeting purposes. Focusing on the 4:1 biotin mAb results, when the amount of SAFF is reduced from 5 ug to 4 ug (column 8, lines 7, 8), separation efficiency drops from 85% to 36%. This drop in separation efficiency can, in part, be compensated by increasing SLAMM to 10 minutes (row 7).

The last line of the Table III shows the results for the mouse-goat anti-mouse Fc ferrofluid (G@mFcFF) common capture system. These results show that SLAMM using G@mFcFF results in excellent separations. Note also that with no magnetic mixing (column 10) 80% of target cells are separated.

In addition to those experiments shown in Example I, several experiments were done in the presence of red blood cells (RBC) to determine the effect of hematocrit level and, thus, whether the SLAMM approach is suitable for separating targets from whole blood or diluted whole blood. For hematocrits up to 15%, SLAMM incubations of 5 minutes resulted in separations that were about 5-7% lower yield than in the absence of RBC. In those experiments, the level of mAb was the same as the lower level used in Table I, viz. 0.3 ug/mL of cells. Also, yields could be improved by extending the time of magnetic separation to 15 minutes. These observations show that simultaneous incubations of all components of an indirect protocol cell separation (mAb, common capture, and cell mixture) can be performed in the presence of RBC and that conditions or concentrations of mAb, common capture agent, and separation can be optimized (such as raising levels of reagents moderately) to obtain satisfactory yields.

### Example 2

### SLAMM-5 Isolation of CD3+ Cells from Buffy Coat Prepared from Human Blood

A buffy coat was prepared from 5 mL of blood drawn from a healthy young adult male and resuspended to 1 mL. From a lysed aliquot, it was determined that 2.5×10⁷ PBMC was recovered and the hematocrit was 25%. We then divided this sample into 0.5 mL aliquots, added 0.15 ug of biotinylated anti-CD3 (0.7 biotin/Ig) and 22 ug SAFF to each, immediately mixed by vortexing, and then applied the SLAMM protocol for 5 minutes. The mixtures were diluted 5-fold and separated in a quadrupole for 10 minutes, after which supernatants were removed and magnetically recovered cells were resuspended in fresh buffer for an addition round of separation. This wash step was repeated and visual inspection indicated that the recovered cells appeared to be free of RBC. Recovered cells were treated with PE-labeled anti-CD3 (Tonbo Biosciences, San Diego, Calif.), washed free of unbound reagent, and analyzed by flow cytometry (Amnis Flowsight, Seattle, Wash.). From the flow cytometry analysis, it was determined that recovered CD3+ cells were on average of 97.4% pure and that the yield was 19%. Considering that about 30% of PBMC would be expected, this corresponds to a 63% yield which is quite reasonable in view of the fact that 30% of the anti-CD3 mAb is unlabeled. It is reasonable to assume that a higher level of biotin conjugation (likely 2-3/Ig) would lead to greater yields and that further optimization of reagents would also contribute to greater recovery. This example illustrates the potential of the SLAMM protocol disclosed herein and its inherent advantages. It is noteworthy that this entire procedure—from preparation of the buffy coat to final recovery of CD3+ cells took less than 40 minutes. With the addition of density enhancers to the initial blood sample that would result in a buffy coat with considerably less RBC contamination, this time could be further reduced because less washes of recovered cells would be required to free CD3+ targets of RBC.

It is very important to note that results disclosed here are not just consequences of the magnetic manipulations to which samples have been subjected. It is believed that there is a more fundamental mechanism operable here. This is best illustrated in Table III which gives the results for experiments in which there was substantially simultaneous incubation of components using the indirect cell separation protocol without magnetic/vortex mixing employed (see last column). These results are for total incubation times of only 5 minutes prior to magnetic separation. For the biotin-streptavidin systems, 64-73% efficiency of separation was obtained and for the mAb-G@mFcFF protocol the efficiency was 80%. These results are surprising given that mAb incubations alone normally take 15-20 minutes and likely would take even longer at the concentrations of mAb employed in these experiments (0.27-0.55 ug/mL).

These results suggest that there might be underlying mechanisms that promote these reactions. One avenue we explored was to ask the question: does the presence of magnetic nanoparticles in some way increase binding of components in a reaction? This inquiry was prompted by some oddities of magnetic nanoparticles observed microscopically. For example, when we have created stable aggregates of our ferrofluids such that they can be barely observed with light microscopy, we have observed what appears to be a coordinated oscillating motion of these aggregates. This led to the question: does just the presence of ferrofluid lead to mixing at the molecular level. To examine the possibility that something like this might be occurring, we performed experiments to look at whether the binding kinetics of mAb to T cells was affected by ferrofluid. To do this, T cells were mixed with anti-CD3 in the presence of a non-specific protein coated ferrofluid. The sample was split and one portion was subjected to the 40 second magnetic/vortex mixing described above for the course of the experiment. The other sample was left on the lab bench away from any magnetic gradients. A control of mAb plus cells and no ferrofluid was also set up on the bench away from any magnetic gradients. At intervals (0, 4, 6, 8, 10, 15 and 20 minutes), samples of each mixture were withdrawn, diluted 5-fold with buffer, centrifuged to remove unbound mAb, and treated with fluorescently labeled goat anti-mouse antibody. Thus, by quantitating fluorescence the comparative binding kinetics can be determined. The results were identical, suggesting that the presence of ferrofluid alone in a reaction does not alter binding of mAb to T cells.

There are many reasons why the simultaneous incubation protocols we have disclosed here can be beneficial. In the case of the mAb-G@mFcFF system, when a mAb binds to cells, the Fc region is presented on cell surfaces in a spatially advantageous manner. As a result, a G@mFcFF nanoparticle approaching that bound mAb has a 1 in 2 chance of colliding with an Fc region as opposed to a 1 in 3 chance were the mAb free in solution. In addition, the bound mAb will contribute to enhanced binding kinetics because it is anchored to a target and will likely rotate less. Consequently, cell-bound mAb may well favor binding of this particular capture agent. A mAb that first binds to G@mFcFF also has stereochemical advantages in binding to cell antigens since the antigen binding regions of the mAb are arranged so that they are directed away from the nanoparticle. SAFF binding to biotins on biotin-mAb Fc regions positions mAbs in a favorable orientation, as the Fab portions are projected out and away from the nanoparticle, which would be expected to promote productive interactions. There are likely other mechanisms in action, and we offer these as possibly a few of many.

The SLAMM approach confers several important advantages including time savings and reduced reagent usage. Thus, by eliminating the effort required to create target-specific reagents and the efficiency that SLAMM offers in the labeling and capture of specific entities, this methodology has useful applications beyond positive selection. SLAMM would be ideal for performing negative selections, in which case all cells except those desired are bound and removed during separation. This approach is useful in cases where naïve cells, i.e., cells that have not been altered or activated by the separation process, are desired. For example, if a naïve population of CD4+ helper cells is required, PBMC can be exposed to a cocktail of mAbs directed to all non-naive CD4+ cells followed by the removal of such cells by targeting them with a common capture magnetic nanoparticle and then performing subsequent magnetic separations. Such mAb cocktails are well known in the art and typically consist of mAbs that bind CD8, CD14, CD16, CD19, CD20, CD36, CD56, CD66b, CD123, TCRγ/δ, glycophorin A, and/or CD45RO. Where naïve CD8+ cells are desired, PBMC would be treated with a cocktail containing mAbs targeting CD4, CD15, CD16, CD19, CD34, CD36, CD56, CD123, TCRγδ, and/or CD235a which would then be bound by an appropriate common capture magnetic nanoparticle for separation. Likewise, the SLAMM approach can be applied to separation of various other cell populations and subpopulations from peripheral blood cell and bone marrow cell, including CD34+ stem cells, CD19+ B cells, CD14+ monocytes, CD15+ granulocytes, and CD56+ natural killer cells.

In applying SLAMM to the above systems where numerous cell types are targeted for removal, there are several strategies that could be employed. A cocktail of appropriate mAbs biotinylated at levels of 3-5 biotins per molecule could be added simultaneously with streptavidin ferrofluid to a cell suspension or, alternatively, the mAb cocktail can be added after streptavidin ferrofluid addition, as no reaction of the latter with cells will have occurred. Likewise, a non-biotinylated system could be employed where, for example, the ferrofluid bears an anti-mouse Fc mAb that targets two or four determinants on the Fc region. Provided there is sufficient excess capacity of the ferrofluid component, the potential of mAb-ferrofluid aggregates is minimal. In this manner the ferrofluid nanoparticles formed in situ will be as multi-specific as the number of specific mAbs used. Alternatively, each mAb and an appropriate level of ferrofluid can be dosed sequentially into the mixture, followed by magnetic manipulations to promote formation of conjugates until all the mAbs of the cocktail have been added. In addition, the method can be applied to reactions where cell concentrations vary substantially, from as low as 5 target cells per mL to as high as 1×10⁸ target cells per ml. Thus, the SLAMM method is well-suited for the enrichment of low-frequency populations, including CD34+ stem cells and circulating tumor cells of epithelial nature. Separation of circulating tumor cells can be accomplished with mAbs directed to EpCAM (CD326), mammaglobin, cell-surface vimentin cell-surface (CVS), and other cell surface markers on cells derived from solid tumors but not expressed on cells of hematopoietic origin (Sieuwerts et al. JNCI J Natl. Cancer Inst. (2009) 101(1): 61-66).

As noted earlier, the SLAMM methodology does not seem to be altered by the order in which the key reagents (viz., cell sample, mAb, and common capture agent) are added, provided these reagents are mixed rapidly. On the other hand, it is clear that in some situations it might be advantageous to thoroughly mix the cell sample and the common capture agent before the reaction is initiated. Non-specific binding of a common capture agent to cells (e.g., interaction with Fc receptors) is easily dealt with by means well known in the art, including using appropriate cell buffer solutions or pre-incubating cells with plasma and FcR-specific antigens. Once mixed, such a sample could either have appropriate mAb added and mixed or, alternatively, the sample could be aliquoted and different mAbs added and mixed, thus affording the opportunity to do multiple specific cell separations for the same cell sample. In either case, the samples would be SLAMM treated and moved to a magnetic gradient separation module for recovery, subsequent wash steps, and retrieval of the positive or negative population as desired.

As disclosed above and shown in Table III, there is an advantage to performing SLAMM at relatively high target cell concentrations so as to take advantage of the law of mass action and collision probabilities. For a biotin-streptavidin system, mAbs conjugated with 1-4 biotins/molecule would be employed. As noted above, an ideal mAb-biotin conjugate would be a Fab fragment where biotin is placed on the end the molecule opposite from the antigen binding site. There are a variety of ways to accomplish that, one of which, as mentioned, is to place biotin on a linker arm of the hinge sulfhydryl of a Fab′.

For a secondary antibody common capture agent such as one directed to the Fc region of the labeling mAb, there are numerous choices. High affinity goat, sheep, donkey, etc. anti-Fc antibodies are readily available, as are rat mAb directed to Fc epitopes and isotype specific antibodies. Using isotype specific common capture agents has advantages since it allows for captured cells to be subsequently labeled with different isotype-specific antibodies that would enable identification. Again, as noted above, an ideal antibody based common capture agent would comprise Fab fragments of those antibodies mentioned above that are linked to capture particles such that the binding sites of those Fab fragments project away from the particles.

As should be evident for this example, the rapidity with which SLAMM can be performed would improve throughput over existing systems since incubation times are substantially reduced. In addition, the economical use of mAb that SLAMM allows gives this approach major cost and performance advantages. SLAMM eliminates the wasteful step in other protocols where mAb is discarded in the supernatant after cells are labeled and washed before common capture agent addition; hence, the SLAMM system provides the most efficient use of mAb for cellular separations.

### Example 3

### Prophetic Example a Clinical Scale CD3+ Cell Isolation Employing SLAMM

In the past few years, it has become evident that cells of the immune system can be manipulated and/or engineered to attack and destroy tumor cells (see for example: Lizee et al, Annu Rev Med 2013, 64: 71-90). Based on recent successes of such procedures with several blood cancers and the potential for extending this approach to solid tumor treatments, there is need for rapid and relatively inexpensive systems for large scale (clinical-scale) cell separations. The economical use of mAb and magnetic reagents makes SLAMM an ideal protocol for such cellular isolations.

Where it is desired to engineer T cells (CD3+ cells), this could be accomplished in a very straight forward manner using SLAMM. Starting with an apheresis product of 10¹⁰ cells in a volume of about 1.0 L, the first step is to remove platelets (known to interfere with immunomagnetic separations). This can be done by centrifugations at appropriate g. forces or by well-known methods employing membrane technology (e.g. spinning membrane filtration: Fresenius Kabi AG and Fresenius Kabi USA, Lake Zurich, Ill. USA), after which cells would be brought to a volume of about 80 mL with an appropriate buffer containing FcR-blocking reagents (that do not react with any of common capture agents) as well as DNAse, protein such as human serum albumin (HSA), and other proprietary reagents known to reduce non-specific binding. To that cell suspension, a common capture ferrofluid (or some suitable magnetic material) that has linked to its surface an anti-Fc antibody, preferably a Fab fragment attached to the nanoparticle via a site on the Fab fragment distal to its combining site. Fab or Fab′ fragments would ideally be directed to Fc determinants and could be isotype specific. Additionally, the Fab or Fab′ fragments could be derived from various species or be a mAb, such as a rat anti-mouse Fc. The nanoparticle—anti-Fc mAb complexes will be engineered in such a way as to not interact with the FcR blocking reagents, thus eliminating nonspecific nanoparticle-cell binding. The complexes could then be added to 80 mL of cells in a relatively concentrated form, e.g. 5 mL containing (based on results shown in Table III) 4 mg of ferrofluid, mixed thoroughly and followed by addition of 30 ug of anti-CD3 mAb and a volume of buffer such that the final volume of the cell suspension is 100 mL. Mixing of these components and subsequent SLAMM could be done in cylindrical soft plastic tubes having dimensions of about 2.5 cm in diameter and 19 cm tall, thus capable of containing about 100 mL. Such a container could be constructed so as to maintain sterility and be compatible with sterile containers used for preparing the platelet-free cell suspension. The advantage of the soft plastic tube cylindrical container is that it can be placed or have placed against it a magnetic gradient that will cause ferrofluid to be moved in one direction to affect the SLAMM process, yet it is amenable to a simple form of redistributing, i.e. mixing of the ferrofluid following a magnetic dwell. Redistribution or mixing can be accomplished by merely moving the cylindrical container through appropriately placed opposing rollers. In this manner, the SLAMM protocol can be applied to a relatively large volume of sample. Clearly a variety of other vessels and ferrofluid redistribution means could be employed, the most important principle being the maintenance of sterility of the contents.

Given the batch nature of the SLAMM process in this example, separation is best done batch-wise so as to treat each cell suspension uniformly. To effect separation, the SLAMM processed sample would be streamed into a separation chamber which might have the following dimensions: 15 cm×30 cm×0.7 cm. The cells would then be pulled magnetically to one of the 15 cm×30 cm surfaces. During streaming into the separation chamber, the sample would also be diluted 1:3 such that the final volume is close to 300 mL and the cell concentration to about 3×10⁷ cells/mL with target CD3+ cells in this example being about 1×10⁷ cells/mL—conditions that are known to give excellent yields and purities. Following filling of the separation chamber, the chamber would be brought into contact with a magnetic plate gradient array having outside dimensions of about 23 cm×35 cm. There are a variety of well-known magnetic gradient devices which draw magnetic entities to a surface that are suitable in this invention.

In this example of CD3+ cell isolation from 10¹⁰ cells, it would be anticipated that about 3×10⁹ cells would be isolated. If cells are loaded into the chamber at 3×10⁷ cells per mL in a volume of about 300 mL, then in 1.0 cm² surface of collection where the chamber depth is 7 mm, the volume is 700 ul. This translates to 2.1×10⁷ cells in that volume and approximately 30% are expected to be T cells (6.3×10⁶ cells). From theoretical calculation and experimental observations, a monolayer of magnetically labeled cells contains about 1.4×10⁶ cells/cm². For this example, that averages to about 4.5 layers of cells. Depending on the levels of entrapped bystander cells that can be eliminated by filling and emptying the chamber while it is adjacent to the gradient field and while targeted cells are held on the separation surface by magnetic forces, it may not be necessary to perform further purification. From preliminary experiments on model systems, it appears that meniscus ‘scrubbing’ during the buffer wash cycles may be adequate for achieving sufficient purity. Alternatively, the collection chamber can be removed from the magnetic gradient array, resuspended and separated again. In the event that cells are not entrapped and that minimal wash buffer is required to be passed through the chamber to remove non-target cells, the process could be done in less than 25 minutes. Provided that the platelet removal and reconstitution of cells to the appropriate starting condition can be done in 15 minutes, it should be possible to process T cells in less than 1 hour. That is considerably faster than currently competing systems.

The time savings, process cost savings, reagent savings and versatility of the SLAMM procedure disclosed here have the potential to be of very substantial research and commercial value. The time savings in regards to cell separations translate into improved cell viability and cell integrity as well as higher throughput—an important economic factor. Reagent savings are also considerable since the levels of mAb used here are substantially lower than what would be used in indirect protocols and less than those used in most direct assays where a nanoparticle-mAb conjugates are generated—another time-consuming conjugation process. For example, a typical mAb-ferrofluid conjugate would require about 10 ug of ferrofluid and about 3 ug of mAb to form the reagent. From Table III, it is clear that about 10 fold less mAb can be used for the SLAMM protocol disclosed here. Given the costly nature of mAbs that is truly a significant cost savings.

Another benefit, mentioned earlier, is that this disclosure in essence shows that an indirect separation protocol (minimally a two-step process) can be converted in situ to the equivalent direct process and completely obviates the need for the traditional 2-step method and the method of not washing out mAb (Liberti U.S. Pat. No. 5,186,827). Moreover, it appears that simultaneously combining all reagents (labeling mAb, common capture agent, and cell mixture) is significantly advantageous since binding pairs are positioned in stereo-chemically favorable orientations that promote more reactions. Based on these findings, it would be most advantageous to construct labeling reagents and common capture agents that are designed with stereochemical considerations in mind. In the examples disclosed here pertaining to the goat anti-mouse Fc common capture ferrofluid, mAb binds to cells to form a capture target with Fc-regions in a very favorable orientation. We have reason to believe that even more favorable reagents can be created. For example, in a biotin-streptavidin system, if instead of using intact mAb, a Fab′ construct of that mAb is used, such that the hinge sulfhydryl of that Fab′ is linked an extended linker arm which terminates with biotin, then Fab′ bound to cell determinants project biotins in an orientation favorable for binding to a streptavidin common capture agent. In the case of an anti-Fc reagent coupled to a particle, it would be highly preferable to couple the Fab′ (or Fab with appropriate chemistry) onto the particle via, for example, the hinge sulfhydryl which can be done using a variety of well-known chemistries. In that way, a common capture particle is created where all the anti-Fc binding sites are very favorably oriented and optimally positioned such that collisions of that particle with mAb bound to cells will be highly productive. From these principles, it would be clear that all binding pairs used in such reactions will benefit by considering stereochemical factors in the synthesis and construction of such reagents.

It should be clear that the substantially simultaneous reactions of targeting component and common capture reagent as a means of labeling cells, or other entities, is not in any way limited to magnetic nanoparticles. There are a variety of other applications where it is desirable to target some entity onto cells, whether the objective is to make the cell denser, less dense, fluorescent, provide some other modification. Rather than make direct conjugates of such entities, it would clearly be advantageous to use at least some of the principles disclosed here even without using the magnetic mixing enhancement reaction disclosed here. Clearly such reactions can be performed simultaneously subject to the limitations disclosed herein and are beneficial because of the stereo-chemical favorability, kinetic advantages, time savings and economics.

### Example 4

### Prophetic Example of Using Simultaneous Addition of mAb and Common Capture Agents for the Stimulation of T Cells In Vitro

June et al. (U.S. Pat. No. 8,637,307) demonstrated that engineered cells bearing anti-CD3, or anti-CD2, as well as a CD28 agonist very effectively induces polyclonal T cell expansion. US 2014/0087462A1 by Sheffold and Assenmacher reviews the evolution of the invention of the '307 patent for causing polyclonal T cell proliferation via the aforementioned stimulators when they are attached to flat and spherical surfaces, where the latter are of various diameters and surface properties. They further demonstrate that dextran-iron nanoparticles that result from the Molday process (U.S. Pat. No. 4,452,773) are very capable of acting as T cell proliferators when either or both anti-CD3 and anti-CD28 are attached to single iron-dextran microspheres or when appropriate mixtures of microspheres that bear the respective antibodies are used. A more straightforward, economical, and versatile means for accomplishing such stimulations would be to use the disclosure of this invention where reagents that specifically bind CD3 and CD28 and an appropriate common capture agent are simultaneously added to T cells. In that way, combinations of anti-CD3 and anti-CD28 as well as any other relevant monoclonals could be used, thus allowing the requisite T cell stimulation to take place in situ.

One approach for accomplishing this is to mix a sample of T cells with a common capture agent such as a ferrofluid to which has been coupled a rat mAb directed to antibody Fc region determinants, i.e. a common capture agent that recognizes 2-4 sites on mouse Fc region or sufficiently low numbers of determinants such that ferrofluid-mAb clustering is eliminated or minimized. Clearly, an appropriately chosen polyclonal could be used and this method need not be restricted to anti-Fc capture or an antibody-bound capture agent (e.g., the streptavidin-biotin system used herein would also suffice). Given that there is no reaction between the added common capture agent and T cells, immediately following their combining, or at some desired interval thereafter, appropriate ratios of anti-CD3 and anti-CD8 mAbs would be added leading to mAb reactions with T cells and mAb reactions with common capture ferrofluid where such complexes would subsequently react with T cells. Another method would be to employ anti-CD3 and anti-CD28 of different isotypes and pair those reagents with common capture agents with specificity for the individual mAb isotypes—thus effectively creating two nanoparticles in situ each bearing only one kind of anti-CD. Based on the Sheffold and Assenmacher disclosure, these methods using appropriate ratios of mAbs will lead to T-cell stimulation. Because of the simplicity and versatility of these methods, as well as GMP considerations for the reagents (all reagents can be filter sterilized), significant cost reduction can be achieved for both the purveyor and the user. This example also provides the user with latitudes not heretofore available.

### Example 5

### Stimulation of CD3+ Cells by SLAMM with Anti-CD3 and Anti-CD28 mAbs Plus Rat Anti-Mouse IgG1 Ferrofluid

PBMCs were isolated from human blood via the OptiPrep method (Axis Shield, Dundee, Scotland) by combining whole blood with 1.25 mL of Optiprep per 10 mL of blood and centrifuging for 30 min at 1,500 rcf. The PBMC layer was resuspended at 10⁷ cells/mL and moved into a T25 culture flask. Anti-CD3 and anti-CD28 mAbs of the IgG1 isotype (Tonbo Biosciences, San Diego, Calif.) were added (at final concentration of 0.1 μg/mL for each antibody) and a common-capture ferrofluid comprising rat anti-mouse IgG1 was added (at a final concentration of 10 μg/mL) to the PBMC suspension. Half of the sample was magnetically mixed by three cycles of exposure to a magnetic gradient for 30 seconds with subsequent agitation. This aided the formation of magnetic conjugates necessary for T cell activation. The other half of the sample was not magnetically mixed. To determine T cell content, the PBMC preparation was analyzed for the presence of the CD3 marker using flow cytometry (FlowSight, Millipore, Billerica, Mass.). Based on that analysis, the cell mixtures were diluted with ImmunoCult XF T-cell expansion medium (StemCell, Vancouver, British Columbia, Canada) supplemented with 100 IU/mL IL-2 (Gibco, Gaithersburg, Md.) to a final T cell concentration of 10⁶ cells/mL. In parallel, PBMCs were activated with ImmunoCult Human CD3/CD28 T cell Activator (StemCell, Vancouver, British Columbia, Canada) according to manufacturer specifications.

On day 0, human PBMCs contained 0.79% CD25+ cells, as determined by flow cytometry using anti-CD25-PE mAb (Ancell, Bayport, Minn.). On Day 3, we determined that 75.4% of the cells from the magnetically mixed samples were CD25+ and had expanded 3-fold. On Day 7, 93.3% of cells from the magnetically mixed samples were CD25+ and had expanded 5-fold, which was comparable to the ImmunoCult-activated sample data. The non-magnetically mixed and stimulated PBMC samples experienced lower activation and expansion rates than the magnetically mixed samples.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. The supported, mixed metal oxide catalyst, its methods of preparation and use can in alternate embodiments, be more specifically defined by any of the transitional terms “comprising”, “consisting essentially of” and “consisting of”.

1. A method of forming a bioconjugate, said method comprising:
(i) combining substantially simultaneously in a biologically compatible medium (a) a target bioentity having at least one characteristic determinant, (b) at least one targeting agent, each targeting agent comprising multiple binding units, each binding unit having at least one binding site and at least one recognition site, said targeting agent being effective to bind specifically through said at least one binding site to said at least one determinant of said target bioentity to yield a labeled bioentity, and (c) a nanoparticle-borne capture agent having at least one binding moiety that binds specifically to said at least one recognition site of said labeled bioentity, thereby forming the bioconjugate, said nanoparticle-borne capture agent having a physical property rendering the formed bioconjugate differentiable in said medium, each binding unit of said targeting agent(s) having about 1-10 recognition sites present thereon, each capture agent having about 1,000 to 8,000 binding sites per nanoparticle, with the number of binding moieties on said capture agent being at least two fold greater than the average number of recognition sites on all binding units of said targeting agent in said medium; and
(ii) subjecting the medium including said target bioentity, targeting agent and capture agent to incubation conditions of temperature and time effective to promote formation of the bioconjugate, said temperature being in the range of about 0-44° C. and said time being in the range of 3-30 minutes.

2. The method of claim 1, wherein said target bioentity is selected from the group of eukaryotic cells, prokaryotic cells, components of said cells, viruses and proteins.

3. The method of claim 1, wherein said targeting agent comprises at least one antibody which binds specifically to the characteristic determinant of said target bioentity, wherein said antibody is optionally a monoclonal antibody.

4. (canceled)

5. The method of claim 3, wherein said antibody comprises a member of a specific binding pair, said capture agent comprises the other member of said specific binding pair and the formation of said bioconjugate results from binding between the members of said specific binding pair.

6. The method of claim 1 in which a single step of subjecting medium to incubation conditions is performed.

7. The method of claim 1, wherein said capture agent comprises a magnetically responsive material.

8. The method of claim 7 further comprising exposing said medium to a magnetic field gradient to cause aggregation of the bioconjugate.

9. The method of claim 8 further comprising terminating exposure of said medium to said magnetic field gradient and thereafter dispersing said aggregated bioconjugate in said medium.

10. The method of claim 1, wherein said capture agent comprises a fluorescent substance.

11. The method of claim 1, wherein said medium has a pre-determined density and said capture agent comprises a material having a density different from said pre-determined density.

12. The method of claim 11, wherein said capture agent has a density higher than said pre-determined density or has a density lower than said pre-determined density.

13. (canceled)

14. The method of claim 1, wherein the target bioentity comprises eukaryotic cells, at a concentration in said medium of 5 to 1×10⁸ cells/ml and is selected from populations and subpopulations of peripheral blood cells and bone marrow cells selected from the group of CD34+ stem cells, CD3+ T cells, CD4+T helper cells, CD8+T cytotoxic cells, CD19+ B cells, CD14+ monocytes, CD15+ granulocytes, CD56+ natural killer cells and circulating tumor cells.

15. (canceled)

16. (canceled)

17. The method of claim 1, wherein said targeting agent is selected from the group targeting CD34, CD3, CD4, CD8, CD19, CD14, CD15, CD20, CD22, CD24, CD28, CD56, EPCAM, vimentin and mammaglobin.

18. (canceled)

19. The method of claim 1, wherein said binding moiety of said nanoparticle-borne capture agent is selected from the group of avidin, streptavidin, neutravidin, anti-mouse Fc, anti-mouse IgG1, protein A, protein G, anti-phycoerythrin, anti-fluorescein isothiocyanate.

20. (canceled)

21. The method of claim 1, wherein said bioconjugate is formed from a target bioentity comprising CD3⁺ cells (T cells), a targeting agent comprising anti-CD3⁺ monoclonal antibody and a nanoparticle-borne capture agent comprising a nanoparticle-bound anti-Fc antibody.

22. The method of claim 21, further comprising exposing said medium to a magnetic field gradient to cause aggregation of the bioconjugate.

23. The method of claim 22, further comprising terminating exposure of said medium to said magnetic field gradient and thereafter dispersing said aggregated bioconjugate in said medium.

24. The method of claim 1, wherein each targeting agent present in said medium has 3-5 recognition sites present thereon.

25. The method of claim 1, which is performed batchwise.

26. A method for clustering two or more cell surface receptors to effect activation of said cell by forming a bioconjugate between said receptors and two or more monoclonal antibodies and capture nanoparticles by substantially simultaneous combination thereof.

27. The method of claim 1, wherein said bioconjugate is a stimulated T cell bioconjugate, said method comprising:
(i) combining substantially simultaneously in a biologically compatible medium, CD3⁺ cells (T cells), an anti-CD3 antibody, an anti-CD28 antibody and a magnetic nanoparticle-bound anti-Fc antibody, the concentration of anti-CD3 antibody, and anti-CD28 antibody in said medium being in the range of about 0.05 to about 1.5 μg/ml, the concentration of magnetic nanoparticle-bound anti-Fc antibody in said medium being equal to or greater than the anti-CD3 antibody concentration;
(ii) subjecting the medium including said CD3⁺ cells, anti-CD3 antibody, anti-CD28 antibody, and magnetic nanoparticle-bound Fc antibody to incubation conditions of temperature and time effective to promote formation of the stimulated T cell bioconjugate, said temperature being in the range of 10-42° C. and said time being in the range of 5-30 minutes;
(iii) exposing said medium to a magnetic field gradient to form a stimulated T cell bioconjugate aggregate;
(iv) terminating exposure of said medium to said magnetic field gradient and thereafter dispersing said stimulated T cell bioconjugate aggregate in said medium.

28. A method of separating a subpopulation of cells of interest from a mixed cell population containing same, said subpopulation of cells having at least one characteristic determinate, said method comprising:
(i) combining substantially simultaneously in a biologically compatible medium, said mixed cell population, at least one targeting agent, each targeting agent comprising multiple binding units, each binding unit having at least one binding site and at least one recognition site, said targeting agent being effective to bind specifically through said at least one binding site to at least one characteristic determinate of said subpopulation of cells, thereby yielding a labeled cell, and a nanoparticle-borne capture agent having at least one binding moiety that binds specifically to said at least one recognition site, thereby forming a bioconjugate comprising said subpopulation of cells, said combination being contained in a non-magnetic containment vessel having a wall surface in contact with said medium, each binding unit of said targeting agent(s) having about 1-10 recognition sites present thereon, each capture agent having about 1,000 to 8,000 binding moieties per nanoparticle, with the number of binding moieties on said capture agent being at least two fold greater than the average number of recognition sites on all binding units of said targeting agent in said medium; and
(ii) subjecting the medium including said mixed cell population, targeting agent and capture agent to incubation conditions of temperature and time effective to promote formation of said bioconjugate, said temperature being in the range of about 0-37° C. and said time being in the range of 3-30 minutes;
(iii) positioning the vessel containing the formed bioconjugate adjacent to an external high gradient magnetic field generator, operable to attract said formed bioconjugate to the wall surface of said vessel and immobilize said bioconjugate thereon;
(iv) removing the vessel contents other than the immobilized bioconjugate;
(v) recovering the formed bioconjugate comprising said subpopulation of cells of interest, wherein the recovering step optionally comprises immobilizing the formed bioconjugate on the vessel wall surface under the influence of an external high gradient magnetic field applied to said vessel, and
wherein the cell subpopulation is optionally selected from populations and subpopulations of peripheral blood cells and bone marrow cells selected from the group of CD34+ stem cells, CD3+ T cells, CD4+T helper cells, CD8+T cytotoxic cells, CD19+ B cells, CD14+ monocytes, CD15+ granulocytes, CD56+ natural killer cells and circulating tumor cells.

29. The method of claim 28, further including, after the positioning step and prior to the recovering step, washing the immobilized bioconjugate with a wash solution to dislodge therefrom embedded non-magnetic substances.

30. The method of claim 29, further including, after the removing step and prior to the recovery step, replacing the wash solution with a biologically compatible medium, releasing the immobilized bioconjugates from said wall surface and dispensing the released bioconjugates in the replacement medium.

31. The method of claim 30, wherein the washing, replacing and releasing steps are repeated multiple times before the recovering step.

32. (canceled)

33. (canceled)

34. The method of claim 28, wherein:
(vi) said targeting agent is selected from the group targeting CD34, CD3, CD4, CD8, CD19, CD14, CD15, CD20, CD22, CD24, CD28, CD56, EPCAM, vimentin and mammaglobin;
(vii) said nanoparticle-borne capture agent is selected from the group of avidin, streptavidin, neutravidin, anti-mouse Fc, anti-mouse IgG1, protein A, protein G, anti-phycoerythrin, anti-fluorescein isothiocyanate; and
(viii) said cell population is CD3+ T cells, said targeting agent is an anti-CD3 antibody, and the binding moiety of said nanoparticle-borne capture agent is an anti-Fc antibody.

35. (canceled)

36. (canceled)
